(19) United States
(12) Patent Application Publication
(10) Pub. No.: US 2024/0382176 A1
(43) Pub. Date: Nov. 21, 2024

(54) Integrated Electrodiagnostic, Physiological and Ultrasound Systems and Methods

(71) Applicant: Cadwell Laboratories, Inc., Kennewick, WA (US)

(72) Inventors: Abram Gardner, San Clemente, CA (US); Michal Holub, Block 707 (SG); David Jensen, Richland, WA (US)

(21) Appl. No.: 18/665,414

(22) Filed: May 15, 2024

Related U.S. Application Data
(60) Provisional application No. 63/618,198, filed on Jan. 5, 2024.
(60) Provisional application No. 63/502,468, filed on May 16, 2023.

(51) Int. Cl. A61B 8/00; A61B 5/388; A61B 5/395; A61B 8/08; G16H 30/20; G16H 40/67

(52) U.S. Cl. CPC A61B 8/4416; A61B 5/388; A61B 5/395; A61B 8/0841; A61B 8/463; A61B 8/465; A61B 8/54; A61B 8/56; G16H 30/20; G16H 40/67

(57) ABSTRACT

An integrated multi-modality system has a first modality corresponding to an EDX or physiological device, including associated amplifier and different types of stimulators, and a second modality corresponding to an US device, including associated probes and beam former, in data communication with a computing device. The system is configured to synchronize EDX and US devices for time-locked collection and analysis of both the EDX and US data thereby allowing combined functionality, review, and analysis. The system generates an integrated multi-modal data file that combines the processed EDX and/or physiological and US data into a unitary file with a singular format thereby supporting a plurality of clinical applications.

### CROSS-REFERENCE

The present specification relies on U.S. Provisional Patent Application No. 63/618,198, titled “Integrated Electrodiagnostic/Physiological and Ultrasound Systems and Methods”, filed on Jan. 5, 2024, for priority. The present specification also relies on U.S. Provisional Patent Application No. 63/502,498, titled “Integrated Electrodiagnostic/Physiological and Ultrasound Systems and Methods”, filed on May 16, 2023, for priority. The above-mentioned applications are herein incorporated by reference in their entirety.

### FIELD

The present specification is related generally to the field of diagnostic systems. More specifically, the present specification is related to integrated electrodiagnostic, physiological and ultrasound systems and methods.

### BACKGROUND

Several medical procedures involve using multiple sensors on the human body for the recording and monitoring of data required for patient care. Information, such as vital health parameters, cardiac activity, bio-chemical activity, electrical activity in the brain, gastric activity and physiological data, are usually recorded through on-body or implanted electrodes, or more generally sensors, which may be controlled through a wired or wireless link. Typical patient monitoring systems comprise a control unit connected through a wire to one or more electrodes coupled to the specific body parts of the patient.

Neuromonitoring or electrodiagnosis involves the use of electrophysiological methods, such as, but not limited to, electroencephalography (EEG), electromyography (EMG), and evoked potentials, to diagnose the functional integrity of certain neural structures (e.g., nerves, spinal cord and parts of the brain) to assess disease states and determine potential therapy or treatment. Electromyography (EMG) is a low-risk invasive procedure in which a small, insulated needle with an exposed tip, having a known exposed surface area, is inserted through the skin into muscle and is used to record muscle electrical activity of a patient. The needle is incrementally moved within each muscle both axially (in and out) and radially (side to side).

Ultrasound imaging is a test that uses sound waves to create a picture, also known as a sonogram, of organs, tissues, and other structures within the body. An ultrasound can also show parts of the body in motion, such as a heart beating or blood flowing through blood vessels. Conventional ultrasound machines comprise a computer console, video monitor, and an attached transducer, also known as a probe. The transducer is a small hand-held device which is placed on an area of a patient's body that needs to be examined. During an ultrasound imaging procedure, a clinician applies a small amount of gel on an area of a patient's body that needs to be examined and places the transducer on the area. The gel allows sound waves to travel back and forth between the probe and the area under examination. The transducer transmits inaudible, high-frequency sound waves into the body and listens for the returning echoes. The ultrasound image is immediately visible on a video monitor. The processor generates an image based on the loudness (amplitude), pitch (frequency), and time it takes for the ultrasound signal to return to the probe.

Clinicians and researchers can currently perform both electrodiagnostic (EDX), physiological examinations and ultrasound (US) imaging. This is typically achieved using different equipment and performing separate examinations at different times. Conventional systems do not provide integrated EDX and US examinations.

Accordingly, there is need for an integrated system providing the capability of integrated recording, display, and analysis of EDX and/or physiological and US examination data. Further there is a need to automatically correlate EDX data with US data with respect to commonly observed, monitored, or otherwise examined anatomical structures.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses system adapted to concurrently acquire first data and second data of a patient, the system comprising: an electrodiagnostic device configured to acquire the first data indicative of voluntary or stimulated electrophysiological signals from a nerve or muscle of the patient; an ultrasound device configured to apply acoustic energy to the patient and acquire the second data, wherein the second data is indicative of reflected sound from the patient's tissue in response to the application of said acoustic energy into the patient; a unitary handheld device in electrical communication with the electrodiagnostic device and the ultrasound device, wherein the handheld device is adapted to concurrently apply electrical stimulation to a first anatomical location of the patient and said acoustic energy to a second anatomical location of the patient and wherein the handheld device is further configured to acquire the first data and the second data; a circuit comprising a clock adapted to generate a time, wherein the circuit is further configured to receive and synchronize the first data and the second data using said time in order to generate third data; a computing device in data communication with at least one of a) the electrodiagnostic device and the ultrasound device and b) the handheld device, wherein the computing device includes a processor and memory storing a plurality of programmatic instructions which when executed by the processor, configures the processor to: acquire the first data, the second data, and the third data; analyze the first data, the second data, and the third data; and generate one or more graphical user interfaces in order to display the first data, the second data, and the third data based on a selection of at least one of a plurality of clinical applications.

Optionally, the third data comprises a unitary file having both the first data and the second data in a same format.

Optionally, the electrodiagnostic device is configured to perform at least one of an electromyography study, a nerve conduction study, an evoked potential study, and a repetitive nerve stimulation study.

Optionally, the plurality of clinical applications comprises a stimulated M-mode study, an electrodiagnostic triggered M-mode study, a stimulated B-mode study, an electrodiagnostic triggered B-mode study, an ultrasound stimulation study, a repetitive nerve stimulation study, a triggered electrodiagnostic study, video synchronization, and an automatic needle tip identification and 3D visualization of ultrasound and electrodiagnostic data.

Optionally, when the plurality of programmatic instructions is executed by the processor, the processor is further configured to: automatically acquire at least a portion of the second data indicative of a current depth of a tip of a needle in the patient; using said second data, cause a display of the current depth of the needle in at least one graphical user interface; cause a display of a window in the at least one graphical user interface, wherein the window is configured to enhance the needle tip and correlate its location in the ultrasound image with relevant electrodiagnostic data; store the ultrasound image with needle position and motor unit potential (MUP) data; and cause a display of said MUP data on the ultrasound image. Optionally, when the plurality of programmatic instructions is executed by the processor, the processor is further configured to extract from the first data said MUP data and cause the display of said MUP data near the needle tip on the ultrasound image.

Optionally, the electrodiagnostic device is an electromyography device.

Optionally, the third data comprises data indicative of a test set-up support for generating a muscle scoring table from an electromyography protocol.

Optionally, the third data comprises data indicative of navigator support in a graphical user interface.

Optionally, when the plurality of programmatic instructions is executed by the processor, the processor is further configured to cause a display of a toggle control adapted to toggle between a first protocol and a second protocol, wherein the first protocol and the second protocol are different.

Optionally, the first protocol corresponds to an electromyography protocol and the second protocol corresponds to an ultrasound protocol.

Optionally, when the plurality of programmatic instructions is executed by the processor, the processor is further configured to generate a dock cine toolbar in the one or more graphical user interfaces.

Optionally, the system is adapted to synchronize a cine buffer and an electromyography buffer.

Optionally, when the plurality of programmatic instructions is executed by the processor, the processor is further configured to cause a display two images side-by-side in the one or more graphical user interface, wherein the two images are indicative of at least two of the first data, the second data and the third data.

Optionally, when the plurality of programmatic instructions is executed by the processor, the processor is further configured to receive a manual selection of two or more images using the one or more graphical user interface, wherein the two or more images are indicative of at least two of the first data, the second data and the third data.

Optionally, when the plurality of programmatic instructions is executed by the processor, the processor is further configured to create report tokens to support image comparisons, wherein the image is indicative of at least one of the first data, second data or third data.

Optionally, when the plurality of programmatic instructions is executed by the processor, the processor is further configured to: cause the ultrasound device to access a computing device storing a list of a plurality of open ultrasound scan orders; cause the ultrasound scan order to be selected from the list of the plurality of open ultrasound scan orders; add the selected ultrasound scan order to a local study list of the ultrasound device; acquire one or more images corresponding to the selected ultrasound scan order; and transmit the one or more acquired images to a storage server.

Optionally, the computing device is a DICOM worklist server and the storage server is a DICOM storage server.

Optionally, when the plurality of programmatic instructions is executed by the processor, the processor is further configured to: receive an ultrasound scan order from a user; and submit the ultrasound scan order to an electronic health record system.

Optionally, when the plurality of programmatic instructions is executed by the processor, the processor is further configured to display the one or more acquired images and link the one or more acquired images to the ultrasound scan order.

The present specification also discloses a system adapted to concurrently acquire first data and second data of a patient, the system comprising: an electrodiagnostic device configured to acquire the first data indicative of voluntary or stimulated electrophysiological signals from a nerve or muscle of the patient, wherein the electrodiagnostic device is configured to perform at least one of an electromyography study, a nerve conduction study, an evoked potential study, and a repetitive nerve stimulation study; an ultrasound device configured to apply acoustic energy to the patient and acquire the second data, wherein the second data is indicative of reflected sound from the patient's tissue in response to the application of said acoustic energy into the patient; a unitary handheld device in electrical communication with the electrodiagnostic device and the ultrasound device, wherein the handheld device is adapted to concurrently apply electrical stimulation to a first anatomical location of the patient and said acoustic energy to a second anatomical location of the patient and wherein the handheld device is further configured to acquire the first data and the second data; a circuit comprising a clock adapted to generate a time, wherein the circuit is further configured to receive and synchronize the first data and the second data using said time in order to generate third data, wherein the third data comprises a unitary file having both the first data and the second data in a same format; a computing device in data communication with at least one of a) the electrodiagnostic device and the ultrasound device and b) the handheld device, wherein the computing device includes a processor and memory storing a plurality of programmatic instructions which when executed by the processor, configures the processor to: acquire the first data, the second data, and the third data; analyze the first data, the second data, and the third data; and generate one or more graphical user interfaces in order to display the first data, the second data, and the third data based on a selection of at least one of a stimulated M-mode study, an electrodiagnostic triggered M-mode study, a stimulated B-mode study, an electrodiagnostic triggered B-mode study, an ultrasound stimulation study, a repetitive nerve stimulation study, a triggered electrodiagnostic study, video synchronization, and an automatic needle tip identification and 3D visualization of ultrasound and electrodiagnostic study.

In some embodiments, the present specification also discloses an integrated multi-modality system comprising: an electrodiagnostic device configured to generate first data indicative of electrical responses in nerves and/or muscles of a patient as a result of application of electrical stimulation; an ultrasound device configured to generated second data indicative of reflected sound from the patient's tissue as a result of injecting sound waves into a patient's body; a handheld stimulator/receiver tool configured to provide a stimulus and concurrently acquires first and second data; a circuit configured to acquire and process the first and second data using a shared clock in order to generate integrated multi-modal data; a computing device in data communication with the electrodiagnostic and ultrasound devices, wherein the computing device includes a processor and memory storing a plurality of programmatic instructions which when executed by the processor, configures the processor to: record and analyze the first, second and integrated multi-modal data; and generate one or more graphical user interfaces in order to display the first, second and/or integrated multi-modal data corresponding to a plurality of clinical applications.

Optionally, the electrodiagnostic device is configured to perform one of electromyography, nerve conduction studies, evoked potential, repetitive nerve stimulation exams.

Optionally, the integrated multi-modal data combines the processed first and second data into a unitary file with a singular format.

Optionally, the plurality of clinical applications corresponds to stimulated M-mode study, electrodiagnostic triggered M-mode study, stimulated B-mode study, electrodiagnostic triggered B-mode study, ultrasound and repetitive nerve stimulation study, ultrasound, triggered electrodiagnostic study, video synchronization, automatic needle tip identification and 3D visualization of ultrasound and electrodiagnostic data.

Optionally, the processor is configured to: automatically detect a tip of a needle; continuously display, in an ultrasound image, a current depth of the needle in a portion of at least one graphical user interface; automatically adjust a focus of display, in the portion of the at least one graphical user interface, to the tip of the needle; display a zoom window in the at least one graphical user interface, wherein the zoom window is configured to show a zoomed-in image of the needle tip; store the ultrasound image with needle position and MUP identification; and display MUP quantification results on the ultrasound image.

In some embodiments, the present specification also discloses an integrated multi-modality system comprising: an electromyography device configured to generate first data indicative of electrical responses in nerves and/or muscles of a patient as a result of application of electrical stimulation; an ultrasound device configured to generated second data indicative of reflected sound from the patient's tissue as a result of injecting sound waves into a patient's body; a handheld stimulator/receiver tool configured to provide a stimulus and concurrently acquires first and second data; a circuit configured to acquire and process the first and second data using a shared clock in order to generate integrated multi-modal data; a computing device in data communication with the electromyography and ultrasound devices, wherein the computing device includes a processor and memory storing a plurality of programmatic instructions which when executed by the processor, configures the processor to: record and analyze the first, second and integrated multi-modal data; and generate one or more graphical user interfaces in order to display the first, second and/or integrated multi-modal data corresponding to a plurality of clinical applications.

Optionally, the integrated multi-modal data provides needle guidance for delivering Botox injection to the patient.

Optionally, the integrated multi-modal data enables test set-up support for generating a muscle scoring table from a standard electromyography protocol.

Optionally, the integrated multi-modal data enables navigator support in a graphical user interface.

Optionally, the system provides toggle hardware controls between first and second protocols corresponding to ultrasound and electromyography.

Optionally, the one or more graphical user interface generates a dock cine toolbar.

Optionally, the system enables synchronization of cine and electromyography buffers.

Optionally, the one or more graphical user interface is configured to display two images side-by-side, wherein the two images are indicative of the first, second or integrated multi-modal data.

Optionally, the one or more graphical user interface is configured to display two images corresponding to one or more calculations, wherein the two images are indicative of the first, second or integrated multi-modal data.

Optionally, the one or more graphical user interface enables manual selection of two or more images for display, wherein the two or more images are indicative of the first, second or integrated multi-modal data.

Optionally, the system enables creation of report tokens to support image comparisons, wherein the image is indicative of the first, second or integrated multi-modal data.

Optionally, the one or more graphical user interface enables ultrasound scanning in a split screen display for comparison purposes.

Optionally, the processor is configured to: enable a user to place an ultrasound scan order in Epic; cause the ultrasound device to point to a DICOM worklist server that lists a plurality of open ultrasound scan orders; cause the ultrasound scan order to be selected from the list of the plurality of open ultrasound scan orders; add the selected ultrasound scan order to a local study list of the ultrasound device; acquire images corresponding to the selected ultrasound scan order; transmit the acquired images to a DICOM storage server; and displays the image in Epic linked to the ultrasound scan order.

Optionally, the system is configured to support HL7 based DICOM image transfer.

Optionally, the one or more graphical user interfaces enable visualization of needle shaft and tip during ultrasound guided needle insertion.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A shows a first view of an integrated multi-modality system, in accordance with some embodiments of the present specification;

FIG. 1B shows a second view of the integrated multi-modality system of FIG. 1A, in accordance with some embodiments of the present specification;

FIG. 1C is a flow diagram illustrating data acquisition, storage, retrieval and display processes enabled by the integrated multi-modality system, in accordance with some embodiments of the present specification;

FIG. 1D is a screenshot showing a multi-modal data file, in accordance with some embodiments of the present specification;

FIG. 1E is an exemplary GUI (graphical user interface) generated by a multi-modality exam module or engine for enabling a user to find and open a multi-modal data file, in accordance with some embodiments of the present specification;

FIG. 2 is an exemplary GUI generated by the multi-modality exam module or engine, in accordance with some embodiments of the present specification;

FIG. 3 is an exemplary GUI generated by the multi-modality exam module or engine, in accordance with some embodiments of the present specification;

FIG. 4A is an exemplary GUI generated by the multi-modality exam module or engine, in accordance with some embodiments of the present specification;

FIG. 4B is an exemplary GUI generated by the multi-modality exam module or engine, in accordance with some embodiments of the present specification;

FIG. 5 shows toggle hardware controls, in accordance with some embodiments of the present specification;

FIG. 6 shows a dock cine toolbar, in accordance with some embodiments of the present specification;

FIG. 7 is another exemplary GUI generated by the multi-modality exam module or engine, in accordance with some embodiments of the present specification;

FIG. 8 is another exemplary GUI generated by the multi-modality exam module or engine, in accordance with some embodiments of the present specification;

FIG. 9 is another exemplary GUI generated by the multi-modality exam module or engine, in accordance with some embodiments of the present specification;

FIG. 10 is another exemplary GUI generated by the multi-modality exam module or engine, in accordance with some embodiments of the present specification;

FIG. 11 shows an exemplary GUI of a needle shaft and tip visualization tool, in accordance with some embodiments of the present specification;

FIG. 12 is a flowchart of a plurality of exemplary steps implemented by the multi-modality exam module or engine with reference to automatic needle tip identification, in accordance with some embodiments of the present specification; and

FIG. 13 is a flowchart of a plurality of steps involved in supporting an ultrasound DICOM workflow, in accordance with some embodiments of the present specification.

### DETAILED DESCRIPTION

In accordance with some aspects, the present specification is directed towards a single integrated system providing the capability of integrated recording, display, and analysis of ultrasound (US) and EDX (electrodiagnostic) or physiological data.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phrascology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application”, “component” or “engine” used in this disclosure may refer to computer logic utilized to provide a desired functionality, service or operation by programming or controlling a general purpose processor. Stated differently, in some embodiments, a module, application, or engine implements and/or is configured to implement a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions. It should be noted herein that each hardware component is configured to perform or implement the plurality of instructions or programmatic code to which it is associated, but not limited to such functions.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

The term “electrodiagnostic (EDX) studies and/or exams”, as used in this disclosure, may refer to a wide variety of different types of clinical examinations that record voluntary or stimulated electrical activity from the brain, nerve, and muscle to access their functionality. EDX studies include, for example, EMG (Electromyography), NCS (Nerve Conduction Studies), EP (Evoked Potential), RNS (Repetitive Nerve Stimulation), and similar examinations. Certain studies and/or exams such as Nerve Conduction Studies (NCS), Repetitive Nerve Stimulation (RNS), and Blink Reflexes, among others, use electrical stimulation to elicit an electrical response in nerves and muscles.

The term “electromyography (EMG)”, as used in this disclosure, may refer to a diagnostic procedure using needles and/or surface electrodes to record the electrical activity from the muscles to assess the health of muscles, nerves, and neuromuscular junctions.

The term “M-mode” used in this disclosure, may refer to an ultrasound diagnostic presentation mode of the temporal changes in echoes in which the depth (D) of echo-producing interfaces is displayed along one axis with time (T) along the second axis; and a motion (M) of the interfaces toward and away from the transducer is displayed. M-Mode may also be referred to as T-Mode, Triggered Mode, or Time Synchronized Mode.

The term “B-mode”, as used in this disclosure, -refers to an ultrasound diagnostic presentation mode that includes a brightness mode image (“B-mode image”), in which reflection coefficients of ultrasound signals (i.e., ultrasound echo signals) reflected from the interested objects in the target object are shown as a two-dimensional image. In the B-mode image, the reflection coefficients of the ultrasound signals on a display are displayed as brightness of pixels.

The term “cine” refers to a movie or sequence of images with a short time interval.

The term “MRN” or “Medical Record Number”, as referred to in the disclosure, may refer to a unique number that is created for each patient visit and/or hospital encounter to uniquely identify the patient and/or encounter through the healthcare system. The MRN is used to uniquely link the patient and any results, tests, and/or exams or the like during that specific hospital visit.

The term “accession number”, as referred to in the disclosure, may refer to a unique identifier that is created for a patient's scheduled procedure, images, and resulting diagnostic imaging report thereby supporting both clinical workflow and billing.

The term “DICOM”, as referred to in the disclosure, is an acronym for Digital Imaging and Communications in Medicine which is a standard to manage and store medical images (and additional related data). The integrated multi-modality system of the present specification is capable of and configured for storing the ultrasound results using this standard.

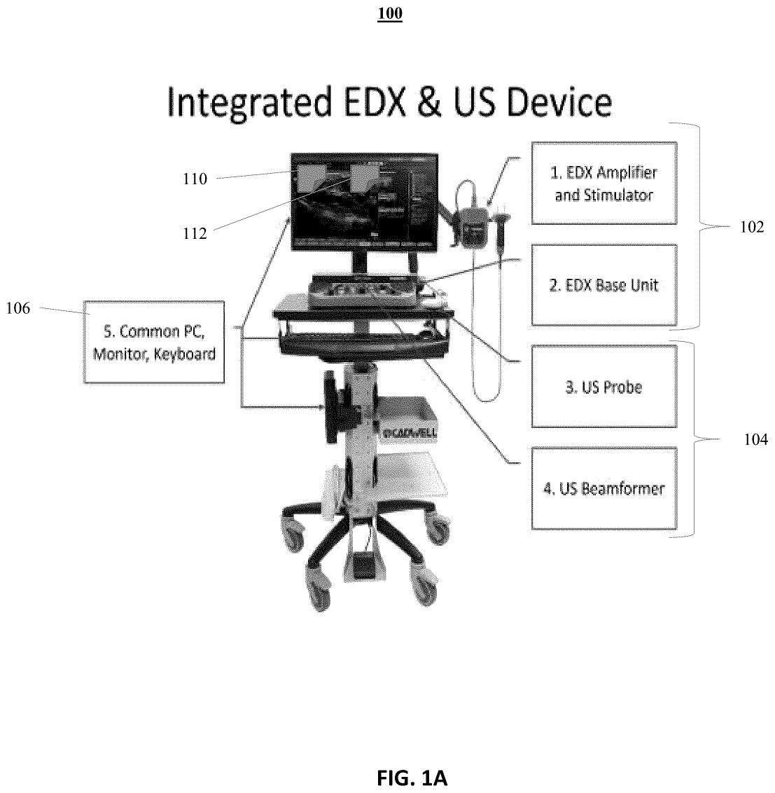

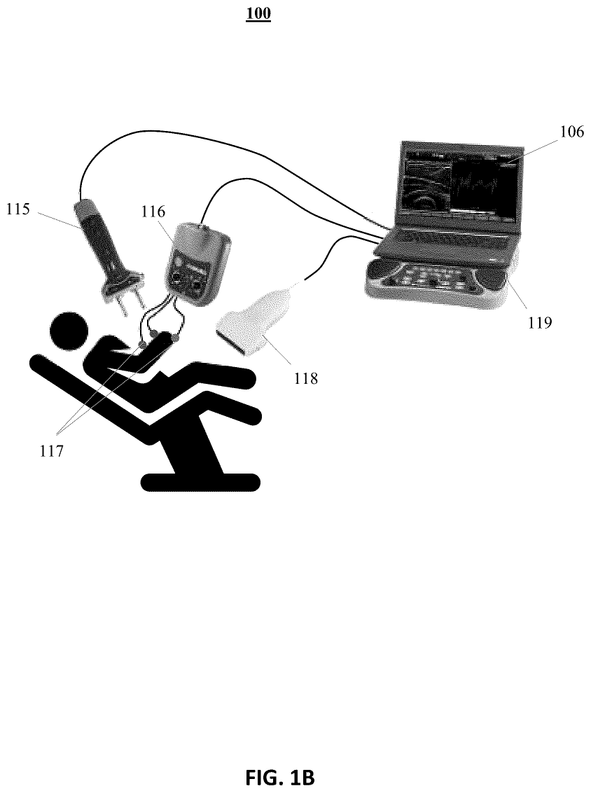

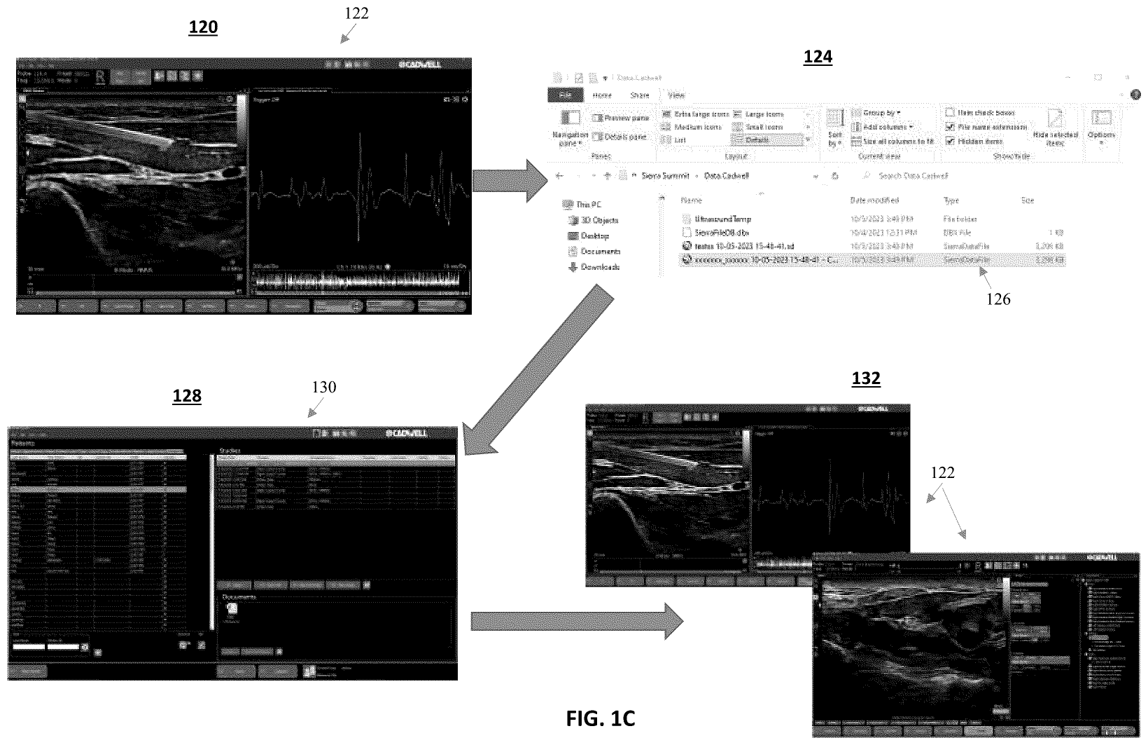

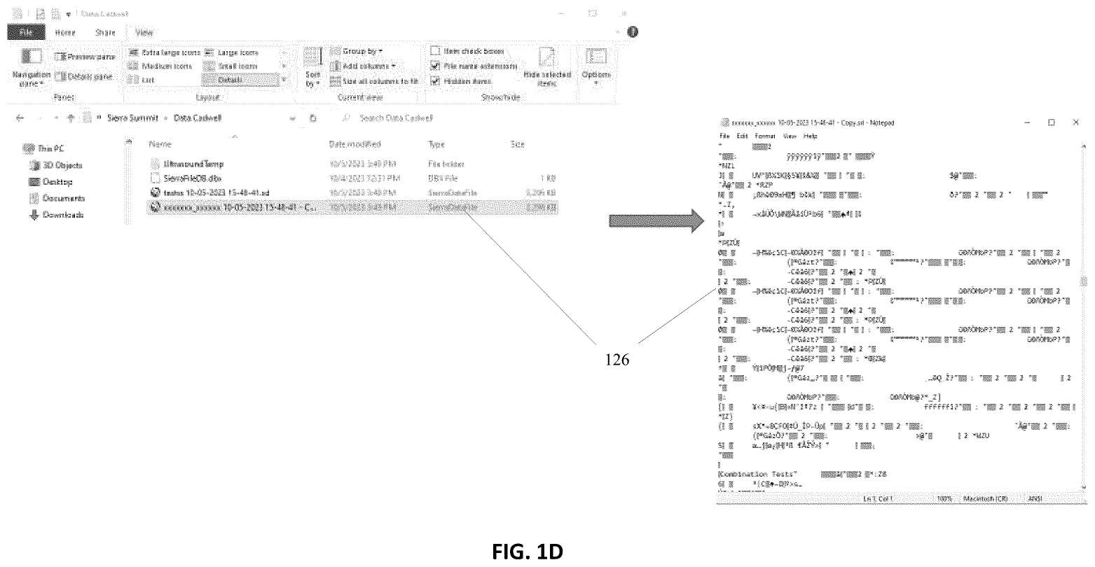

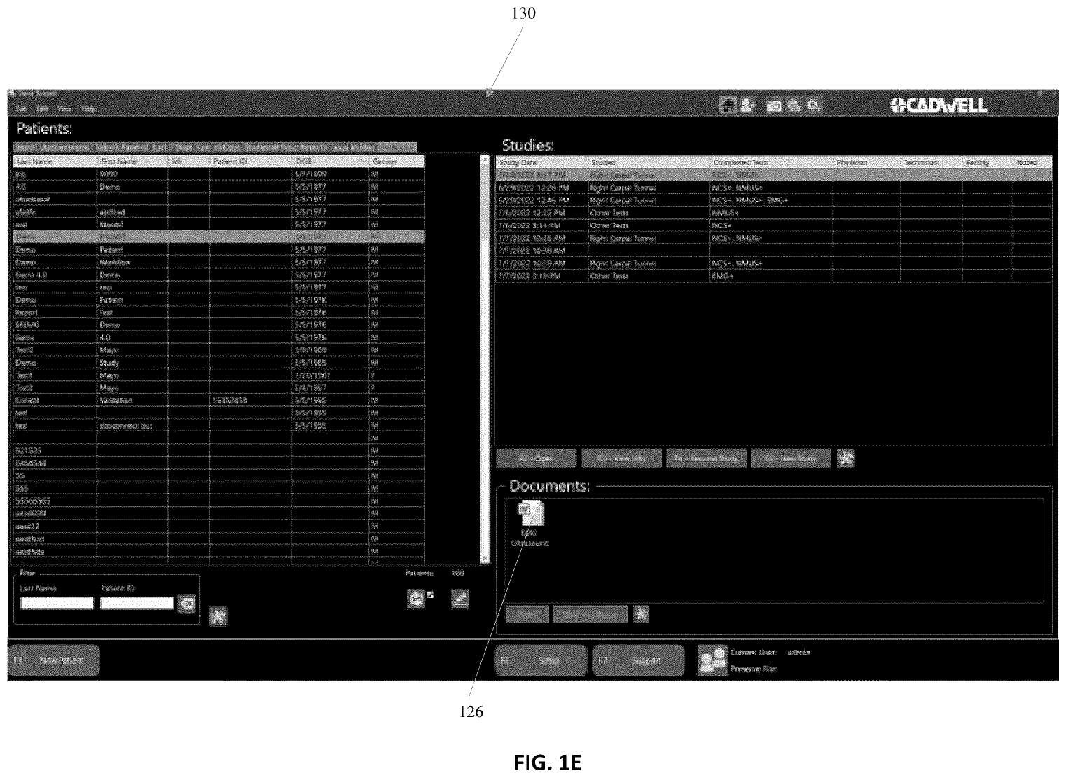

FIGS. 1A and 1B show an integrated multi-modality system 100, in accordance with some embodiments of the present specification. In various embodiments, the system 100 provides combined EDX (electrodiagnostic) and/or physiological devices and capabilities and US (ultrasound, also neuromuscular ultrasound (NMUS)) devices and capabilities. In some embodiments, the system 100 comprises a first modality corresponding to an EDX or physiological device 102, including associated amplifier and different types of stimulators, and a second modality corresponding to an US device 104, including associated probes and beamformer, in data communication with a computing device 106.

As shown in FIG. 1B, system 100 comprises an electrical stimulator probe (also referred to as a ‘StimTroller’) 115 for stimulating a patient's nerves and muscles to evoke different types of physiological signals. In various embodiments, the stimulator probe 115 may be auditory, visual, electrical, or any other type of probe. In some embodiments, the stimulator probe 115 may include a probe such as, but not limited to, a monopolar probe, a bipolar probe and/or a smart probe that may be configured to provide electrical stimulation to nerves and neuronal structures (based on a plurality of stimulation protocols) in order to measure, for example, motor evoked potential (MEP) in response to electrical stimuli. The stimulator probe 115 may also include audio stimulators such as, for example, headphones or a set of earphones in order to measure auditory evoked potential (AEP) or brainwaves in response to auditory stimuli. The stimulator probe 115 may further include visual stimulators such as, for example, glow goggles (having one or more LEDs to generate visual stimuli) in order to measure visual evoked potential (VEP) in response to visual stimuli.

In embodiments, the system 100 further comprises an amplifier 116 to record physiological responses from the patient. In embodiments, the system 100 further includes one or more electrodes 117 that may be placed on the patient and connected to the amplifier 116 in order to acquire physiological responses.

In embodiments, the system 100 further includes an ultrasound probe 118 for recording an ultrasound image/data stream.

In embodiments, the system 100 further includes a dedicated hardware device or base unit 119 which is an integrated control panel (with a dedicated keyboard) configured to control and support the different stimulators, amplifiers, ultrasound and other equipment as necessary. In embodiments, the system 100 further includes a computing device 106 that is configured to implement a plurality of instructions or programmatic code which, when executed, generate auditory, visual and/or electrical stimulation, generate sound waves as well as store and analyze EDX/physiological and US data streams.

In embodiments, an EDX and/or physiological device refers to a device that can perform different types of EDX and/or physiological exams such as, but not limited to, EMG (electromyography), NCS (nerve conduction studies), EP (evoked potential), RNS (repetitive nerve stimulation) studies that use electrical stimulation to elicit an electrical response in nerves and muscles. These responses are recorded using different types of electrodes. In embodiments, an ultrasound (“US”) device refers to a device that can perform ultrasound exams by injecting sound waves into a patient's body and recording and analyzing the reflected sound to gather information about the studied tissue. This information can be displayed and quantified in different ways (B-Mode, M-Mode, Doppler etc.). In some embodiments, the system 100 is configured such that the EDX and/or physiological and US devices can be controlled together.

In some embodiments, the system 100 is configured to receive analog signals from the EDX and/or physiological device 102 and the US device 104 and, process the acquired EDX and/or physiological and US signals or data streams using a shared clock.

In accordance with aspects of the present specification, the computing device 106 includes a synchronization module or engine 110 and a multi-modality exam module or engine 112. The module 110 includes hardware and a plurality of instructions or programmatic code configured to synchronize the EDX device 102 and US device 104. In embodiments, the synchronization refers to the simultaneous and time-locked acquisition/collection and analysis of both the EDX and US data, as integrated multi-modal data, allowing combined functionality, review, and analysis. The module 112 includes a plurality of instructions or programmatic code configured to record, retrieve, display, analyze, and review data from both the exam modalities at the same time.

FIG. 1C is a flow process diagram illustrating data acquisition, storage, retrieval and display processes enabled by system 100, which is configured to perform the described functions, in accordance with some embodiments of the present specification. At step 120, the modules 110 and 112 are configured to enable a user to synchronously acquire first raw data stream corresponding to an EDX and/or physiological modality and second raw data stream corresponding to an US modality. FIG. 1C also shows an integrated GUI (graphical user interface) 122 that is used and configured to enable a clinician to perform EDX and/or physiological exams to collect related first raw data stream and US exams to collect related second raw data stream. In embodiments, the first raw data stream and second raw data stream are processed into processed first data file and processed second data file, respectively. At step 124, the processed first data file and processed second data file are integrated into a multi-modal data file 126, also referred to as third data, which is a unitary file with a binary format for an exam/study. Thus, the Ultrasound and EDX data is rendered and processed simultaneously and stored in the single combined data file 126, such that any post processing done with either data streams updates the combined data file 126. As shown in FIG. 1D, the multi-modal data file 126 (or third data) is generated and stored in the context of a single EDX and/or physiological and US exam with a binary format that is encrypted for HIPPA compliance. It should be noted that, in embodiments, system 100 may not capture a first raw data stream and a second raw data stream to separately generate a corresponding processed first data file and a processed second data file and thereafter combine them to generate an integrated data file. Instead, in embodiments, system 100 may be configured to capture first raw data stream and second raw data stream and integrate the first raw data stream and second raw data stream before processing and generating the multi-modal data file 126.

At step 128, the module 112 is configured to enable the user to retrieve or open the multi-modal data file 126 using a GUI 130. As shown in FIG. 1E, the GUI 130, in some embodiments, is configured to enable the user (with appropriate security credentials) to find, retrieve, and open the data file 126. In some embodiments, the data file 126 can be opened and reviewed in the same GUI in which it was collected, that is, the GUI 122 and GUI 130 may be the same. Alternatively, the GUI 130 may be different from the GUI 122 based on end user preferences and/or the clinical requirement. At step 132, the module 112 renders the multi-modal data file 126 for display in the integrated GUI 122 for simultaneous and synchronized display of the first data and second data.

In embodiments, the system 100 is configured to provide a plurality of clinical applications that are uniquely supported by the unitary multi-modal data file 126 and that would not have been possible by simply taking two separate data files (corresponding to the EDX and/or physiological and US modalities) and matching up the timestamps of those different data files.

In embodiments, the synchronization module 110 and the multi-modality exam module 112 are configured to manage at least one EDX and/or physiological (for example, EMG/NCS) data feed, at least one US data feed, and an optional video data feed from associated acquisition devices (such as 102, 104) and display them concurrently with a synchronized time base so that clinical decisions can be made based on, for example, EMG data, NCS (nerve conduction study) data, US data, quantitative US data (Q Mode) and video data or any combination of these data streams. It should be appreciated that the combination of these data streams allows clinicians to make observation(s) that cannot be obtained from individual data streams since the modules 110, 112 enable correlating electrophysiological muscle and nerve activity with ultrasound imaging and quantification in relation to the specific position of recording electrodes.

In embodiments, system 100 is configured to permit a user (such as, for example, a clinician) to create custom examination types using existing and new EDX and US feature sets allowing them to analyze and measure results across the two modalities. System 100 is, in embodiments, characterized by a plurality of features. In some embodiments a single system that allows for recording of different combinations of EDX or electrophysiological, ultrasound, and video into integrated data streams is provided. In some embodiments, the integrated multi-modal data is stored as a single synchronized entity with the potential to extract clinically correlated information (also referred to as ‘correlated data’) across any modality. In embodiments, the integrated multi-modal data is analyzed to provide currently non-existent clinical information and examination types. In embodiments, the correlated data is combined with the multi-modal analysis along with existing examination results for additional results and clinical conclusions.

In yet another embodiment, the system 100 is configured to enable 3D visualization of US and EDX and/or physiological data. With 3D visualization, a user can select and view images and results (corresponding to US and EDX and/or physiological data) from different view angles in order to identify, for example, the position and extension of compression of a nerve accurately.

In accordance with various aspects of the present specification, the synchronized EDX and/or physiological and US data (also referred to as ‘integrated multi-modal data’ or T-Mode) offers a plurality of benefits and advantages. For example, in some embodiments, the synchronization of the data allows for integration of the EDX and/or physiological and US data streams which enhances clinical analyses by reducing the time of the clinical investigation. By using data from multiple synchronized sources, containing related but independent clinical information, clinicians can focus more quickly and efficiently on pathologies, thus reducing diagnostic time and discomfort to patients. In addition, in some other embodiments, the correlated data provides a platform for exposing clinical markers generated from single data sources that were previously not apparent, which can provide further opportunities for reduced patient clinical evaluation using only one data stream. For example, with ultrasound it is not possible to know if small muscle movements are due to voluntary muscle activation, involuntary fasciculations or involuntary fibrillations. However, synchronized EMG and ultrasound can show the source of the muscle contractions and can help identify even small neuromuscular disorders in the image such as fibrillations and positive sharp waves. Still further, the synchronization of the data allows for expanding the reach and accessibility of clinical examinations to populations that cannot tolerate current electrodiagnostic methods such as neonates, by providing more non-invasive options to clinicians. Still further, the integrated multi-modal data increases the sensitivity and specificity of pathology detection in patients by identifying clinically significant markers in the integrated multi-modal data streams, particularly in the case where non-invasive imaging could provide early detection. In addition, the integrated multi-modal data brings powerful machine learning techniques from image analysis to bear on image data marked using other data streams. Further, the integrated multi-modal data offers clinicians and researchers the ability to create completely new diagnostic examinations and procedures using integrated EDX and/or physiological and US data.

Integrated EDX and/or Physiological and US-Based Clinical Applications

In various embodiments, the system 100 supports a plurality of clinical applications such as, but not limited to, the configurations that will be described in greater detail below.

### Stimulated M-Mode (Also Referred to as T-Mode, Triggered Mode, or Time Synchronized Mode)

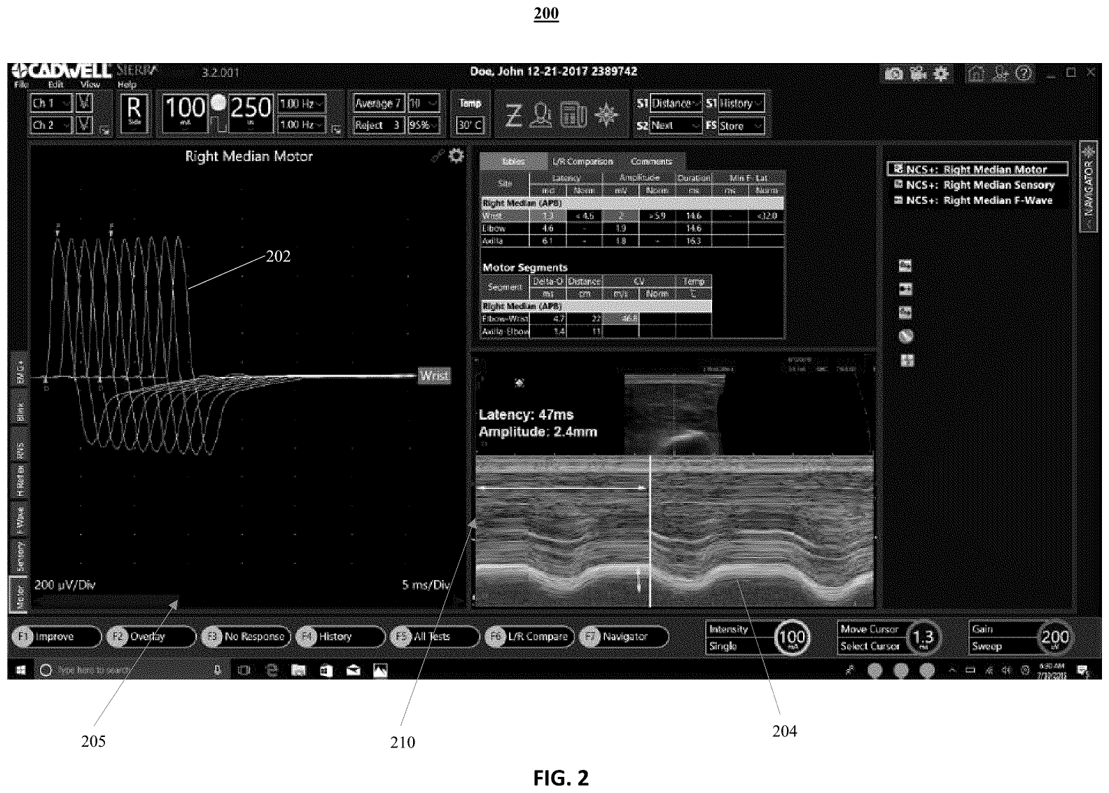

It should be noted herein that while the specification refers to a simulated M-Mode, the term may be used interchangeably with T-Mode, Triggered Mode or Time Synchronized Mode. In some embodiments, the system 100 is configured to enable recording, reviewing, analysis and displaying of data in stimulated M-mode of operation. FIG. 2 shows a first GUI 200 generated by the multi-modality exam module or engine 112 which is configured to display data corresponding to a stimulated M-Mode of operation, in accordance with some embodiments of the present specification. As shown, a first portion 205 of the first GUI 200 displays EMG signals or data 202 corresponding to the muscle response (Compound Muscle Action Potential, CMAP) recorded by surface electrodes placed on top of the muscle (Abductor Pollicis Brevis, APB) while stimulating the right median motor nerve. A second portion 210 of the first GUI 200 displays an M-mode image 204 of US signals or data to enable visual correlation of EMG and US data. Stated differently, referring to FIG. 2, the electrophysiological activity on the left side corresponds to data generated by the contraction of the muscle as recorded by electrodes positioned on top of the muscle while the M-Mode US data (on the right side) represents the same muscle movement as recorded by the ultrasound probe. Thus, two different and independent data streams depict the same event with the potential to confirm findings and provide both quality control and additional information, such as, but not limited to the size of movement and delay in movement. In some embodiments, the stimulated M-Mode of operation is characterized by at least some of the following features: a) provides a capability to record triggered/time synchronized EDX and M-Mode US data; b) allows use of different types of stimulators as triggers—single electrical, dual electrical, auditory, and other stimulators; c) enables measurement of time results such as, but not limited to, latency, duration, or another time-related parameter between time of stimulation and the synchronized representation of the effect, for instance a muscle movement, in the M-Mode data; d) enables quantification of the M-Mode data at specific point(s) in time; for instance, it is possible to measure the largest deflection of a structure in the image; e) allows averaging of M-Mode data; f) enables subtracting two different points of M-Mode data; and g) enables control of the time base of the M-Mode data in a similar manner as the EDX tests.

### EDX-Triggered M-Mode

In another embodiment, an EDX and/or physiological signal triggered M-Mode is available. The EDX triggered mode is triggered by an electrophysiological event, such as the electrical activity from a twitching muscle, in contrast to an operator-controlled event where the operator initiates an electrical stimulation. Thus, the EDX triggered mode allows for the analysis of ultrasound data recorded during a native muscle twitch that is triggered and saved based on the recorded electrophysiological activity from the twitching muscle.

### US-Triggered EDX

In another embodiment, it is possible to acquire US-triggered EDX and/or physiological data, thus, the system is configured to enable collection of EDX data on an event in the US data. For instance, it may be possible to look at twitches in M-Mode (US-generated images), while capturing and averaging EEG data from the motor cortex.

### Stimulated B-Mode

In yet another embodiment, a Stimulated B-Mode, is available and is configured to: a) provide a capability to record triggered B-Mode US data; b) enable placement of markers in the B-Mode data and measure time results such as, but not limited to, latency, duration, or other time-related parameter; and c) enable display of graphs of measurements over time. For instance, an exemplary graph may be displayed for the results of measuring a cross-sectional area of a muscle at different times after an electrical stimulus. Triggered B-Mode offers the ability to have a voltage trigger on an electrodiagnostic signal such as EMG or EKG waveform and associate signals that cross that trigger with a B-Mode image frame. Stimulated B-Mode offers the ability to know which B-Mode frame is associated with a neurophysiologic stimulation (electrical, visual, audible or a combination) and allows the user to make measurements on that image that can be correlated with the stimulation.

### EDX Triggered B-Mode

In some embodiments, system 100 is configured to enable recording, reviewing, analysis and display of data in an EDX triggered B-mode of operation. It should be noted that in EDX triggered B-Mode the actual electrophysiological signal, recorded from a needle or some other type of electrodes, triggers/defines the point in time when the US data is obtained, stored, and analyzed. The time is not controlled by the user such as in Stimulated B-Mode where a user performs an action such as pressing a button but by the recorded physiological activity.

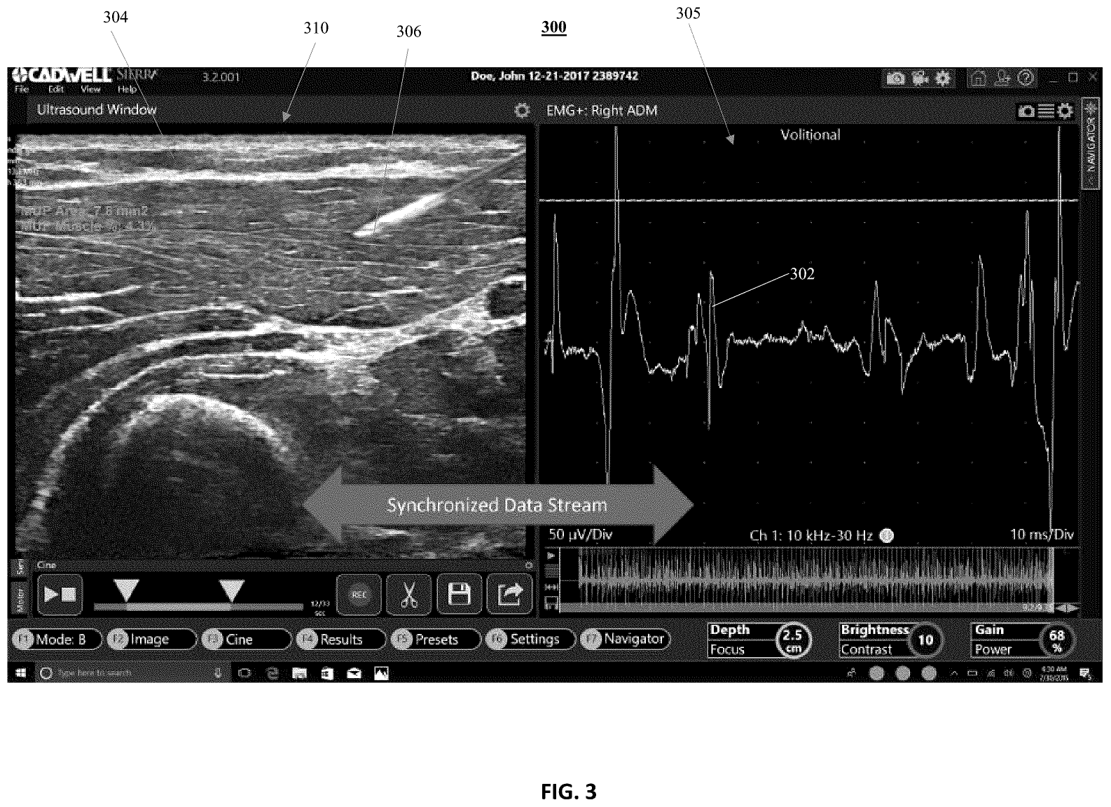

FIG. 3 shows a second GUI 300 generated by the multi-modality exam module or engine 112 to display data corresponding to an EDX signal triggered B-Mode of operation, in accordance with some embodiments of the present specification. As shown, a first portion 305 of the second GUI 300 displays EMG signals or data 302 corresponding to stimulation (using an EMG needle 306) of a patient's abductor digiti minimi muscle (ADM). A second portion 310 of the second GUI 300 displays a B-mode image 304 of US signals or data to enable visual correlation of synchronized EMG and US data.

In embodiments, an EDX triggered T-Mode allows for analysis of the ultrasound data based on electrophysiological activity (that is, T-Mode refers to the mode that allows comparison of electrodiagnostic waveforms and stimulations to US data). For example, a needle is inserted into the muscle (as depicted as 306 in FIG. 3) and the subject is asked to slightly contract the muscle. While contracting the muscle the activity from a specific motor unit can be identified on the right side (the electrophysiological/EDX recording) and different types of analyses can be performed on the US data based on the electrophysiological data. As a non-limiting example, the US data can be averaged on the EDX data.

In some embodiments, the EDX triggered B-Mode of operation is characterized by various features. In some embodiments, for an event that is identified in the EDX and/or physiological data, such as recording fibrillations or MUPs (Motor Unit Potential) using an EMG needle, the US data may be analyzed to determine the extent of the fibrillation or MUP in the US data that is triggered by the specific EMG activity. In embodiments, the EDX triggered B-Mode generates a cine (movie/sequence of images with a short time interval), whereby the movement in time refers to changes between the consecutive images that are perceived as movement by the user. Additionally, creating a sufficient B-Mode image requires multiple overlaid scans which reduces the number of frames per second. In some embodiments, “dual stream” recording is performed and referred to as Q-Mode. In some embodiments, it is possible to collect ultrasound information using two data streams. U.S. patent application Ser. No. 18/183,350, assigned to the Applicant of the present specification, is one such method and is herein incorporated by reference. A first data stream (B-Mode) is controlled by the user (gain, contrast, probe frequency, etc.) while the other data stream (Q-Mode) has predetermined and preset recording parameters and cannot be user-manipulated which allows for a well-defined and reproducible analysis of ultrasound data.

Still further, in some embodiments, the EDX triggered B-Mode of operation supports averaging of US data on the signal trigger, displays MUP measurements in the US image and allows storing the picture showing needle position for a specific recording/MUPs.

### US RNS Study

In yet another embodiment, an application may be a combined US with repetitive serve stimulation. The analysis of US data is a potential option that is less sensitive to electrode location, movement, and other diagnostic challenges. In this embodiments, the system is configured to enable analysis of US data over time to extract sequential information of an anatomical response to nerve stimulation. Referring back to FIG. 2, in embodiments, a user can stimulate a nerve while scanning a muscle with ultrasound. Using M-mode in the ultrasound window or portion 210, the user can measure the time it takes for the muscle to respond to each stimulation and see the amplitude or size of the response, duration of the response, and recovery before the next stimulation is delivered. The comparison of the results, between each stimulation, assists in the diagnosis of neuromuscular function disorders, for example, myasthenia gravis.

### Video Synchronization

In yet another embodiment, the system is configured to enable video synchronization, or the ability to store multiple synchronized video streams. This allows for a number of different clinical, educational, and research applications/functionality. For instance, it allows showing the position of a probe when anomalies are found enabling anatomical localization for surgeons. Video synchronization allows a user to correlate physiologic and ultrasonic data to a physical location of the probe, needle and/or electrodes. For example, one possible use is to measure a change in probe position and correlate that with an ultrasound image so that the images can be stitched together, factoring in both space and time, rendering accurate 3D reconstructions of the anatomy. A synchronized video stream also allows clinicians and surgeons to see the exterior view of the probe, needles, and/or electrodes in relation to the neurophysiologic activity and images that are being measured.

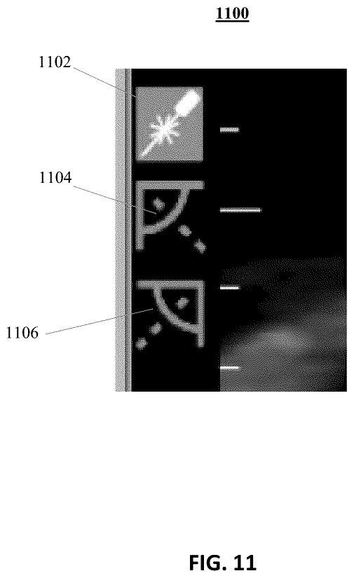

In some embodiments, the integrated multi-modality system 100 of the present specification is configured to enable users to concurrently perform US with EMG and electrical stimulation for needle guidance for the purpose of delivering Botox injections. In some embodiments, the integrated EMG and US system enables a user to better visualize needle shaft and tip during ultrasound guided needle insertion. FIG. 11 shows a GUI 1100 of a needle shaft and tip visualization tool. In embodiments, the needle enhance button 1102 enables the needle enhance feature. In embodiments, the left side needle insertion button 1104 is configured to convey to the multi-modality exam module or engine 112 that the needle is entering from the left side of screen. In embodiments, the right-side needle insertion button 1106 is configured to convey to the multi-modality exam module or engine 112 that the needle is entering from the right side of the screen. The integrated system supports mapping of the tip of the needle on the ultrasound image and uses that mapped location to precisely synchronize the needle location with EDX data and display EDX results with the annotations on the ultrasound image. The system also supports the annotation of the image, on the mapped location of the needle, with information such as the units of botulism toxin or other pharmaceuticals that were injected into that location and/or other EDX result(s) associated with the needle.

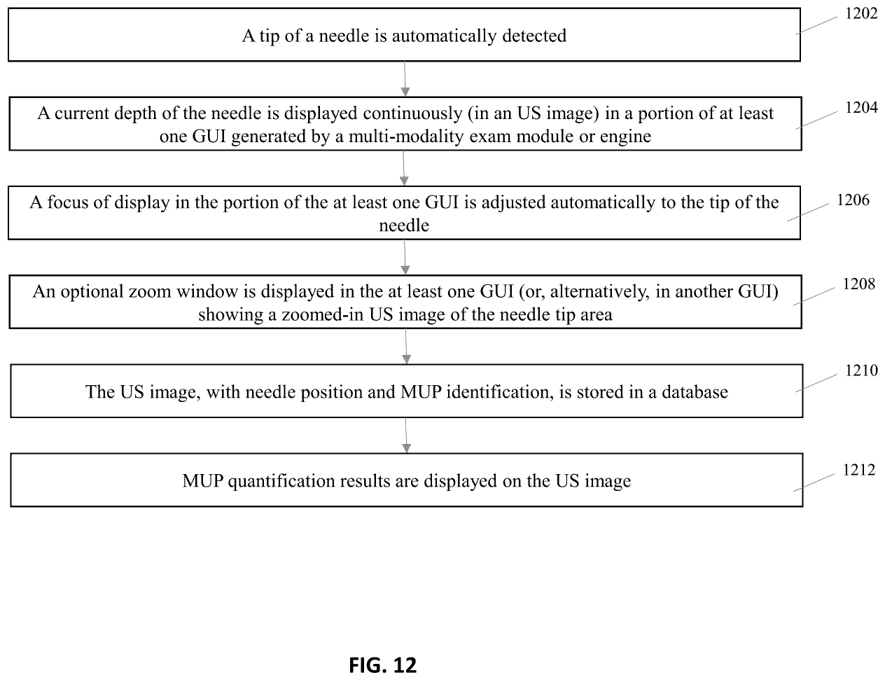

In yet another embodiment, the system may be configured to perform automatic needle tip identification, in which a needle needs to be tracked with high accuracy for various clinical applications. Accordingly, in some embodiments, the system is configured to implement a special needle mode where the multi-modality exam module or engine 112 automatically identifies the needle tip using US data. FIG. 12 is a flowchart of a plurality of exemplary steps implemented by the multi-modality exam module or engine 112 with reference to automatic needle tip identification, in accordance with some embodiments of the present specification. Referring now to FIG. 12, at step 1202, a tip of a needle is automatically detected. This is conventionally done by detecting a needle-shaped object and then looking for an end of that object that may be reflecting backscatter in different directions due to the needle bevel. At step 1204, a current depth of the needle is displayed continuously (in an US image) in a portion of at least one GUI generated by the multi-modality exam module or engine 112. At step 1206, a focus of display in the portion of the at least one GUI is adjusted automatically to the tip of the needle. At step 1208, an optional zoom window is displayed in the at least one GUI (or, alternatively, in another GUI) showing a zoomed-in US image of the needle tip area. At step 1210, the US image, with needle position and MUP identification, is stored in a database associated with the system 100. At step 1212, MUP quantification results are displayed on the US image. It should be appreciated that MUPs are identified in EMG data stream and notated near the needle tip on the Ultrasound. Electrodiagnostic MUP data can also be correlated with Ultrasound RF data stream for the purpose of identifying MUP activity present in ultrasonic RF data.

### Exemplary Integrated EMG and US System Components and Interfaces

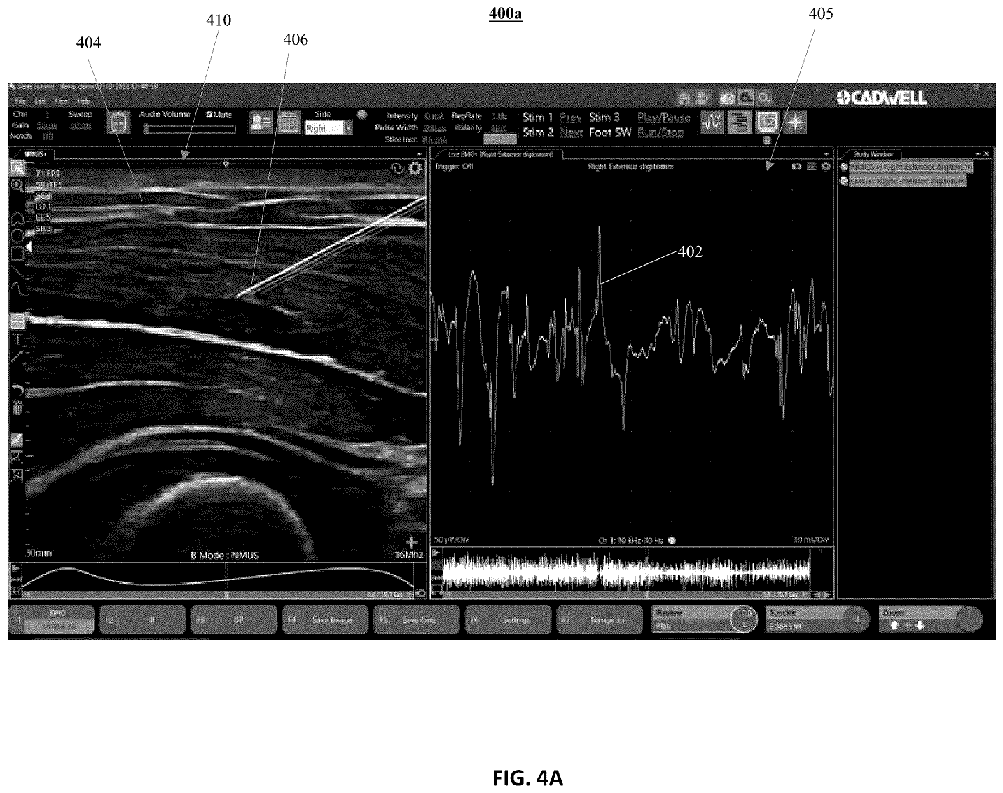

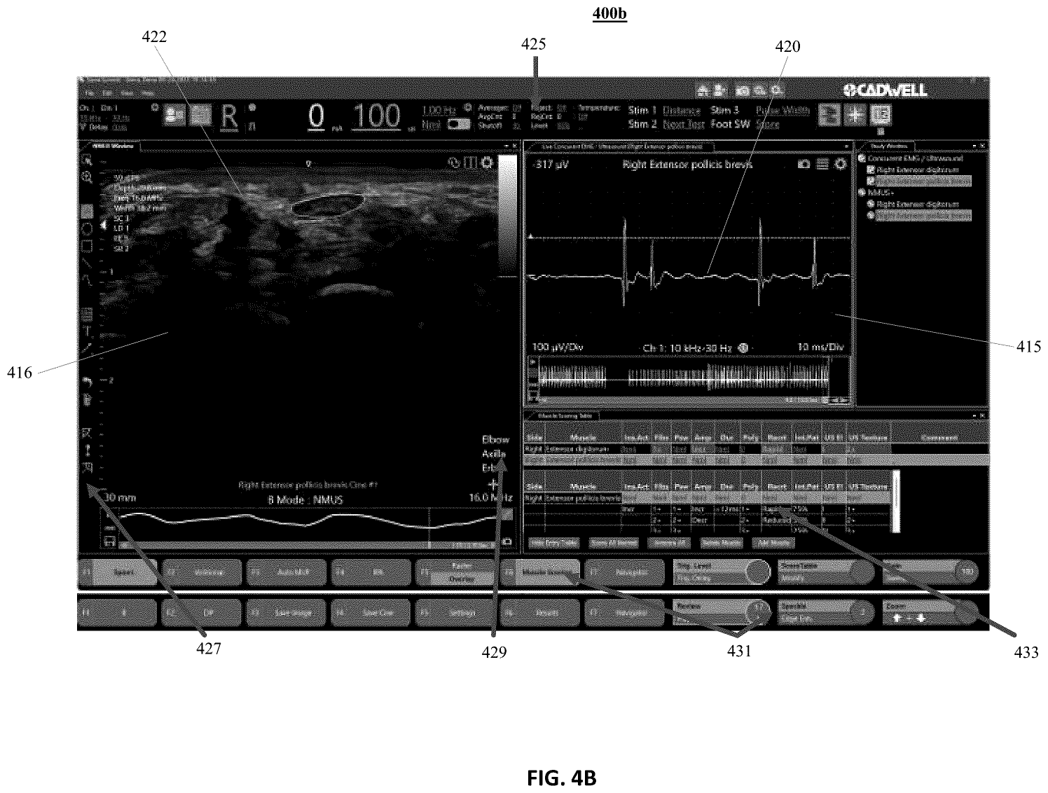

FIG. 4A shows a GUI 400a generated by the multi-modality exam module or engine 112 in order to display an injection needle 406 and EMG data 402, in accordance with some embodiments of the present specification. The GUI 400a displays EMG signal or data 402 and US image 404 side-by-side in first portion 405 and second portion 410, respectively, of the GUI 400. In some embodiments, the integrated EMG and US system is characterized by at least certain features, which will now be described in greater detail.

As a first feature, in embodiments, the integrated EMG and US system is configured to enable test set-up support which allows for the creation of a concurrent protocol that supports both EMG and US features and also allows for a unique muscle scoring table from a standard EMG protocol.

FIG. 4B shows a GUI 400b generated by the multi-modality exam module or engine 112 in order to display both US and EMG data, in accordance with some embodiments of the present specification. The GUI 400b displays EMG signal or data 420 and US image 422 side-by-side in first portion 415 and second portion 416, respectively, of the GUI 400b. A third portion of the GUI 400b is configured to display an EMG/Nerve Conduction toolbar 425 with some EMG/NCV (nerve conduction velocity) related functions such as, but not limited to, channel selection, filter selection, notch filter control, stimulator controls, averaging and any other function that may be necessary. A fourth portion of the GUI 400b displays an ultrasound toolbar 427 with some of the ultrasound related functions such as, for example, zoom, trace tool, ellipse tool, rectangle tool, caliper, curve, annotations, labels, arrows, undo, delete, needle enhancement and any other function that may be necessary. A fifth portion of the GUI 400b displays probe position annotation control 429 that is configured to allow a user to select a currently examined anatomical structure and probe position. A sixth portion of the GUI 400b displays a plurality of mode dependent function keys 431 that change depending on which data window, 420 or 422, has focus thereby providing data-window specific functionalities. A seventh portion of the GUI 400b displays a muscle scoring table 433 which is a user-defined table that allows users to annotate the studied anatomy (muscle or nerve) with their clinical findings-that is, both physiological (for example, Fibs, Amp) as well as ultrasound finding (for example, Echo Intensity, Echo Texture).

As a second feature, in embodiments, the integrated EMG and US system is configured to enable navigator support, wherein a concurrent protocol appears as a test option in a muscles tab, a US tab, and an all tests tab. In addition, selection of a muscle adds both EMG and US muscle to the study window.

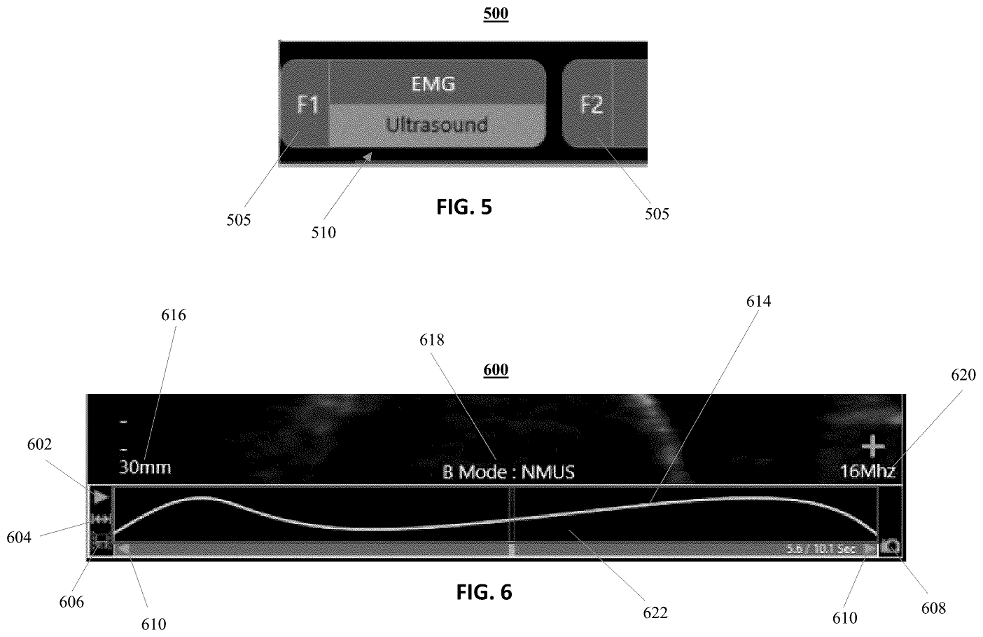

As a third feature, in embodiments, the integrated EMG and US system is configured to provide Toggle Hardware Controls (base unit 119/StimTroller 115 of FIG. 1B) between two test protocols-that is, the integrated buttons on the StimTroller 115 as well as the base unit 119 can be switched between controlling the EMG functionality or the ultrasound functionality. As shown in FIG. 5, in embodiments, the toggle hardware controls 500 include the ability to make F Keys 505 customizable in EMG protocol (just as with US) and to add a toggle option 510 between EMG and Ultrasound base unit controls to F keys, StimTroller Buttons and Footswitch: e.g., F1=EMG/Ultrasound. Thus, referring back to FIG. 1B, the buttons on the StimTroller 115 and the base unit 119 simplify use by providing direct control to different functions of the system by pressing respective buttons on the base unit 119 and the StimTroller 115. For instance, start/stop recording may be supported on both base unit 119 and StimTroller 115. The user can assign their own preferred functions to the different buttons.

As a fourth feature, in embodiments, the integrated EMG and US system is configured to enable a docked cine toolbar, as shown in FIG. 6, in accordance with some embodiments of the present specification. The docked cine toolbar 600 is similar in features and function to the EMG toolbar and the US toolbar (such as the EMG toolbar 425 and US toolbar 427 of FIG. 4B), including the following: i) left side: play back/pause 602, zoom out 604, save cine 606; ii) right side: save image 608, and additional buttons such as, for example, auto position markers, jump to next measurement and other various buttons; iii) dragging in buffer zooms in on a section of buffer, and storing when zoomed in saves only the zoomed-in section, which is the default mouse function (clicking and dragging in the bottom bar 622, displaying the echo intensity trace 614, shows a box that can be dragged out covering a part of the buffer, whereby releasing the mouse button will zoom in on the box content); iv) click and drag in bottom bar 622 or scroll mouse wheel to navigate through cine; v) arrows 610 on either side advance one frame at a time; vi) support EI (echo intensity) min/max trace or graph 614; vii) incorporate adjustable settings similar to EMG+ with selectable text just above cine toolbar; viii) left side: indicates depth 616; ix) center: mode and preset 618 and x) right side: indicates frequency 620.

The visual indicators 616, 618 and 620 indicate text that can be clicked on which will then present different user selectable options.

As a fifth feature, in embodiments, the integrated EMG and US system is configured to enable synchronization of cine and EMG buffers. Thus, i) EMG and cine buffers are synchronized both in real-time (live) and when played back in review; ii) playback of either buffer shall play back both buffers; iii) save cine/Buffer saves both EMG and Ultrasound buffers; iv) option to take snapshot of both EMG and Ultrasound as well as individual; v) option to export combined EMG/Ultrasound buffer to MP4 or Native format.

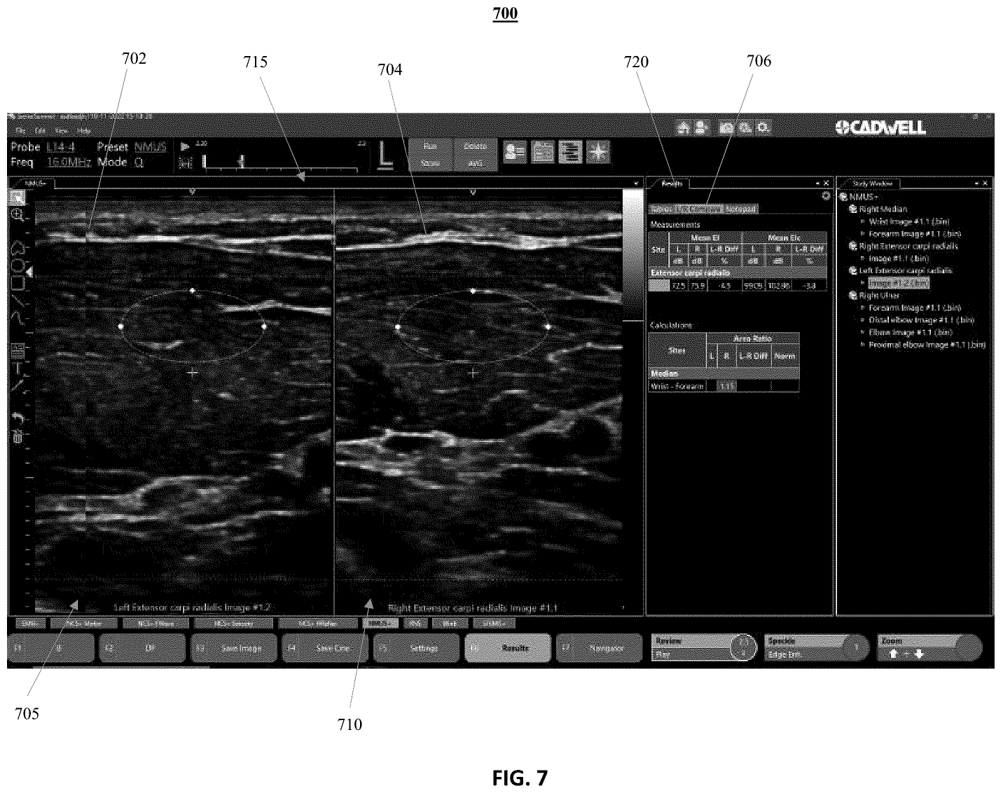

In embodiments, the integrated EMG and US system is configured to enable multi-image display characterized by various features and functionalities. For example, in some embodiments, in the context of the integrated EMG and US system, the multi-modality exam module or engine 112 generates GUIs that enable a user to review images side-by-side. As shown in FIG. 7, the GUI 700 enables a visual comparison of left image 702 and right image 704 in respective first portion 705 and second portion 710 of the GUI 700. The GUI 700 shows both left and right images 702, 704 side by side when L/R Compare Tab 706 is selected in results window or tab data 720. Additional features of the GUI 700 include a) displaying image settings when in Multi-Image Display mode; b) auto centering and cropping images 702, 704 to fit within an image window 715; and c) allowing for each image 702, 704 to be zoomed or resized independently.

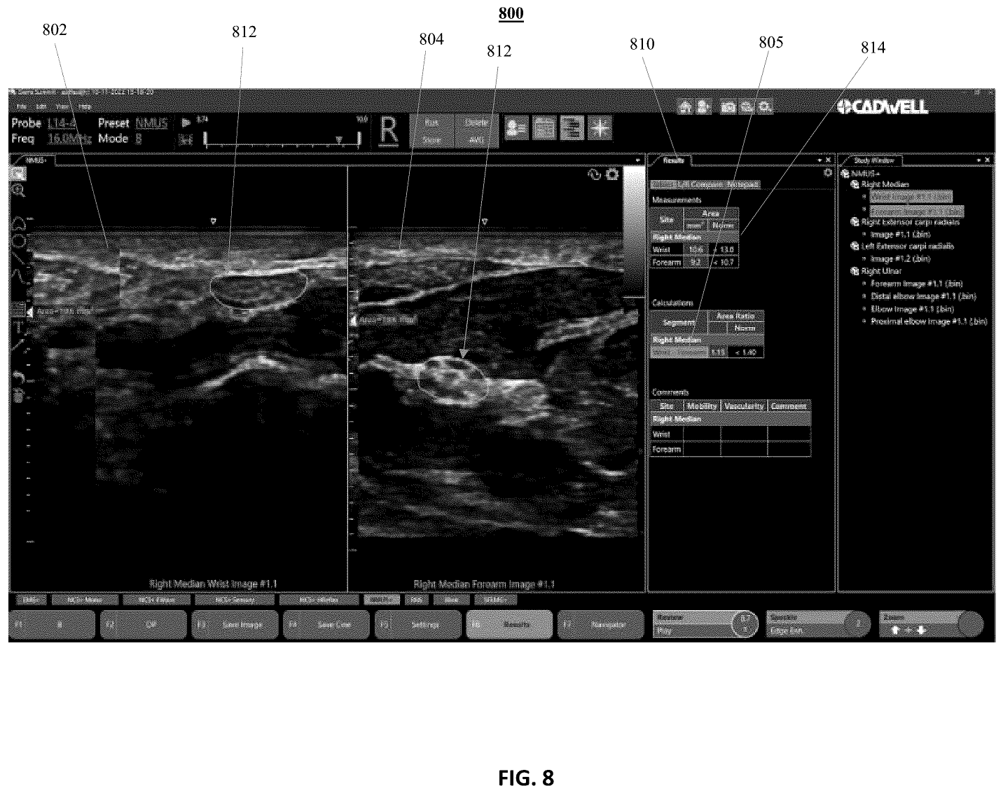

In embodiments, the integrated EMG and US system is further configured to support one or more calculations, as shown in FIG. 8. A GUI 800 shows a first ultrasound image 802 and a second ultrasound image 804 that are used to make measurements by placing different types of markers/cursors on the images (such as, for example, markers 812 for area calculation). In the results window or tab 810, measurement results are stored in table 814 and when the results/calculations 805 are clicked the corresponding images 802, 804, where the calculations were made, are automatically restored.

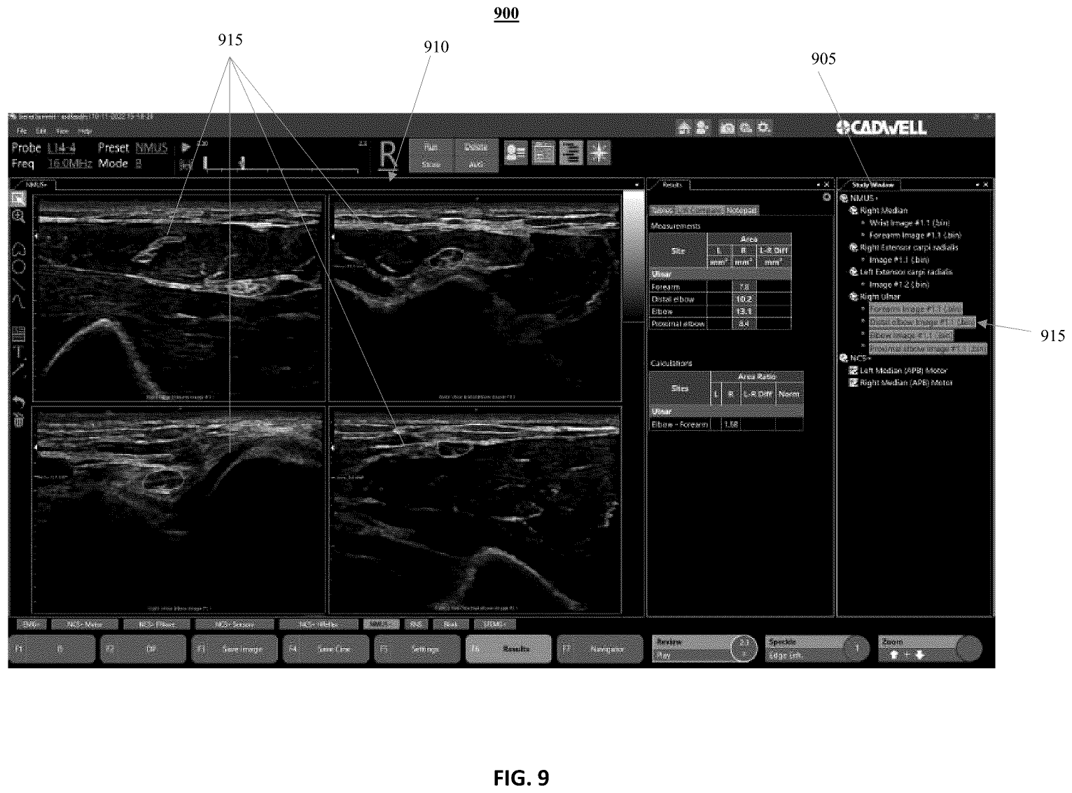

Additionally, in embodiments, the integrated EMG and US system is configured to enable manual select for comparison. As shown in FIG. 9, a GUI 900 illustrates a functionality where a user may hold the CTRL key and select 2 to 4 images 915 from the study window or tab list 905. Additionally, the GUI 900 provides the following features: a) if two images are selected then both images are displayed side by side in an image window 910; b) if three or four images are selected then the images are displayed in 2×2 grid in the image window 910; c) images are auto centered and cropped to fit within the image window 910; and d) each image is allowed to be zoomed independently.

Still further, in embodiments, the integrated EMG and US system is configured to enable the creation of report tokens to support image comparisons. A report token is a variable/link to some of the recorded data that the user can choose to add to an examination report. There are many different report tokens representing the different types of results recorded during the examination that can be added to the examination report. Following are two exemplary report tokens that enable the user to add ultrasound images from the left and the right examined side together with different calculations for side to side comparison of examination results: a) US left/right images (all); and b) US calculation images (all).

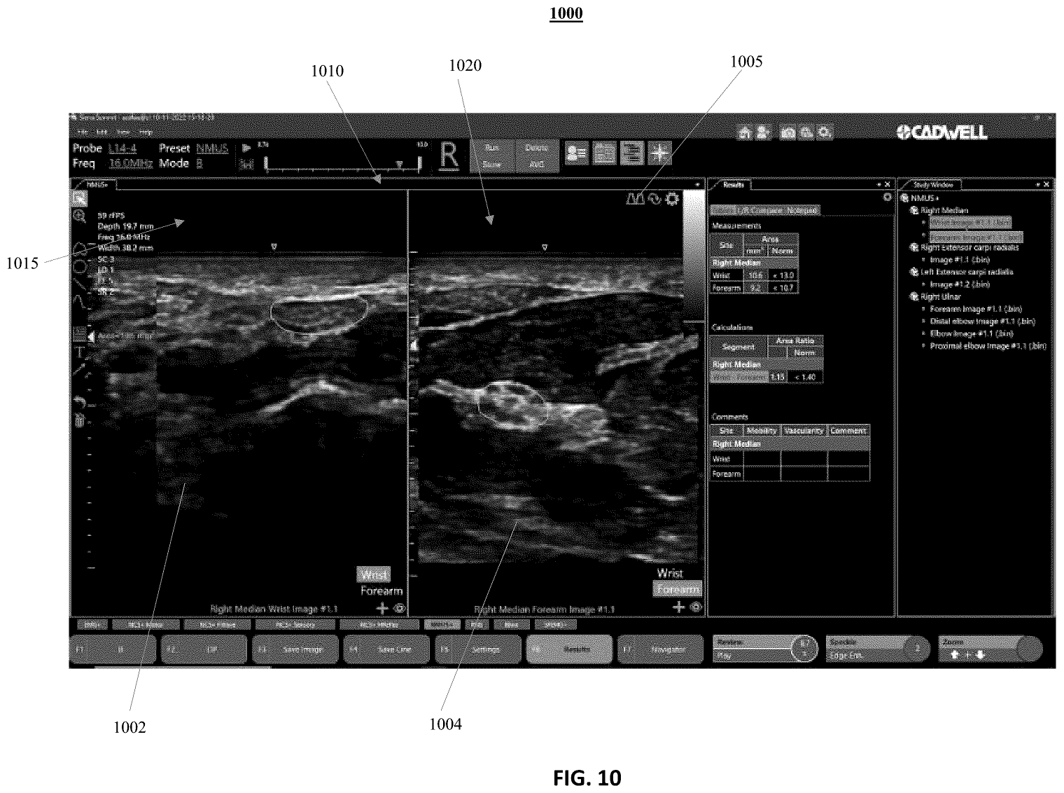

Additionally, in some embodiments, the integrated EMG and US system is configured to enable dual image scanning. In some embodiments, the multi-modality exam module or engine 112 generates at least one GUI that enables a user to scan in a split screen display for comparison purposes. Various features of this embodiment include, for example, supporting live scanning in a dual image display. As shown in FIG. 10, a GUI 1000 displays an Add Dual-Image tool 1005 in a top right corner of the image window 1010. In embodiments, it is possible to freeze/save one image 1002 in a first portion 1015 of the image window 1010 and continue scanning in a second portion 1020 of the image window 1010 for image comparison or extended panoramic view of a structure. In embodiments, the images 1002, 1004 may belong to the same site or different sites/anatomies. In embodiments, the default is to the same site, however the user can select a different site for each image. In embodiments, the dual image scanning also allows for cine support, which has the ability to playback both cines at the same time if a cine is saved.

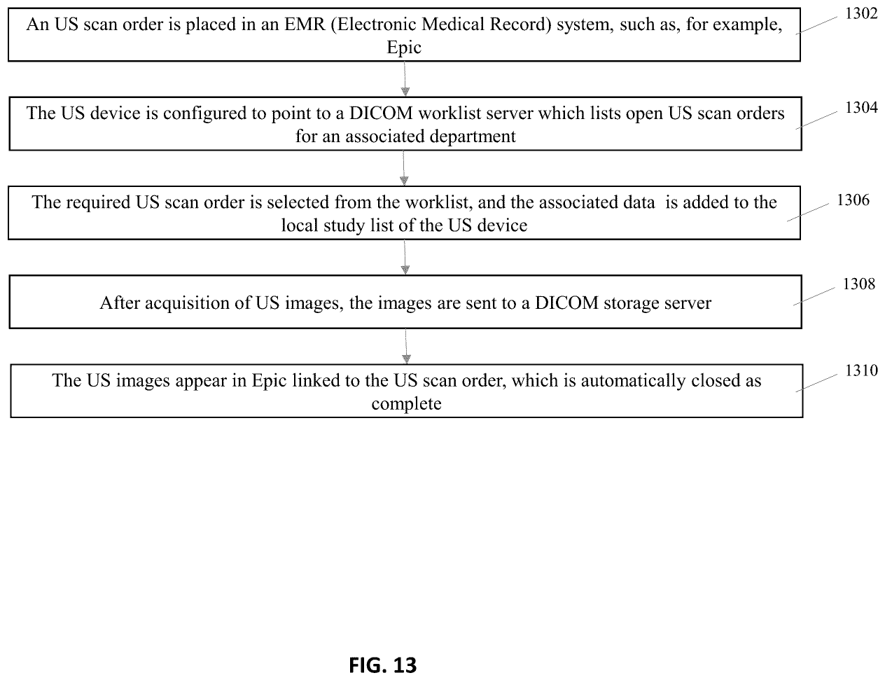

In some embodiments, the integrated EMG and US system is configured to support storage and transmission of DICOM messages, images and cines and other typical DICOM workflows. FIG. 13 is a flowchart of a plurality of steps involved in supporting US DICOM workflow (but does not involve HL7 format), in accordance with some embodiments of the present specification. Referring now to FIG. 13, at step 1302, an US scan order is placed in an EMR (Electronic Medical Record) system, such as, for example, Epic, responsible for storing different types of healthcare information with respect to a patient's hospital visit. The multi-modality system 100 is configured to integrate with EMR systems in order to read scheduled examinations from the EMR systems as well as store results, images, reports back to the EMR systems. At step 1304, the US device is configured to point to a DICOM worklist server which lists open US scan orders for an associated department. At step 1306, the required US scan order is selected from the worklist, and the associated data (such as, but not limited to, MRN (medical record number) and accession number) is added to the local study list of the US device. At step 1308, after acquisition of US images, the images are sent to a DICOM storage server. This step has an option to auto-send all images when a study is closed or manually send all or selected images (also defined in machine configuration). In some embodiments, the US device can optionally be configured to also send DICOM Structured Report (SR) data and allow sending the SR data to a custom database by way of a Mirth server (As known to persons of ordinary skill in the art, Mirth is middleware that connects health information systems so that they can exchange clinical and administrative data). At step 1310, the US images appear in Epic linked to the US scan order, which is automatically closed as complete.

Still further, in some embodiments, the integrated EMG and US system is further configured to support HL7 (Hospital Language 7 Protocol) based DICOM image transfer. As known to persons of ordinary skill in the art, HL7 is a language that is used to communicate different healthcare related information between different systems/devices in a hospital. As a non-limiting example, the HL7 workflow related to the integrated EMG and US device of the present specification may be as follows: a patient is referred for an examination using the integrated EMG and US device. A user requests such an examination in a hospital EMR (such as, for example, Epic) system. This request is transmitted using the HL7 standard containing information regarding the patient (such as, for example, name, length, birthday), the examination to be performed and various other pieces of information. When the physician opens the integrated EMG and US device they can see that the patient is scheduled and that all patient information is already available in the system. After performing the examination, the results are sent back to the EMR (Epic) as well as other healthcare systems (DICOM Servers are used for image storage for instance from ultrasound, MRI, CT etc.) using different protocols (HL7, DICOM).

Also, in some embodiments, the integrated EMG and US system is configured to include results and RF data along with image in DICOM wrapper. As is known to persons of ordinary skill in the art DICOM is a standard supporting both communication between different healthcare systems as well as storage and exchange of data. By storing the ultrasound data both as a static image as well as raw data (RF) and numerical results. The raw data can be further analyzed/manipulated for different purposes such as, for example, for creating new images or measurement results.

In some embodiments, the integrated EMG and US system is configured to enable a user to make manual calculations between measurements in one or more images.

In some embodiments, the integrated EMG and US system is configured to enable a user to select a different measurement for a tool from the image window during an exam.

In some embodiments, the integrated EMG and US system enables a user to perform additional echo intensity measurements to generate histograms that quantify nerve and muscle echotexture. This includes features such as: a) Echo Variance; b) Black/White Ratio for nerves; and measures that show muscle architecture, such as entropy and fat versus muscle data.

In embodiments, the present specification is further directed towards M-mode improvements. Thus, a user can play back and save M-mode scanning with cine; place measurements and annotations on an M-mode image, including: a) create a new time measurement using a caliper tool, b) create a new frequency measurement for M-mode similar to manual frequency measurements in EMG; and c) create a new amplitude measurement for M-mode; and sync M-mode with E Stim and EMG, which includes placing a marker on M Scan horizontal access where a stimulation occurred.

As shown in FIG. 4B, in some embodiments, the integrated EMG and US system generates the GUI 400b in which US function keys 431 are added to a general tab (in a custom buttons section).

In some embodiments, the integrated EMG and US system generates the GUI 400b in which an option is added for specifying the anatomical location at which the Probe is positioned. Selecting an anatomical position (from the Anatomical Position toolbar 429) adds the position to an image label and result table label. The GUI 400b includes Default Probe positions and option for the user to customize and add new positions. An image label example is that of a Right Median Wrist Long Axis. An example of a result table is as follows:

TABLE 1
Result Table.

| | Short Axis | | | | Long Axis | |
|---|---|---|---|---|---|---|
| | Area | | Diameter | | Width | Width 2 |
| Site | (mm²) | Norm | (mm) | Norm | (mm) | (mm) |

Right Median

| | | | | | | |
|---|---|---|---|---|---|---|
| Wrist | 10 | <11 | 1.8 | <32 | 5.5 | 6.3 |
| Forearm | 8 | | 1.5 | | | |

Left Median

| | | | | | | |
|---|---|---|---|---|---|---|
| Wrist | 9 | <11 | 1.7 | <32 | 5.5 | 5.7 |
| Forearm | 8 | | 1.5 | | | |

In some embodiments, the integrated EMG and US system enables the following additional features and characteristics: a) allows the creation of a concurrent protocol (ultrasound and another protocol); b) runs at the same time on a single monitor or two monitors (less common); c) concurrent protocol has two main windows, one for each protocol; d) top and bottom toolbars/base unit controls switch based on which protocol is highlighted; e) in concurrent mode a “view” button on base unit toggles between the two protocols (Toggles base unit control, Top tool bar, F keys and Knobs, highlight border around protocol); f) view button in the top tool bar toggles the views in the specific protocols; g) adding a test protocol to a concurrent protocol makes a copy of that test protocol and leaves the original unchanged; and h) concurrent protocol is launched from the navigator with a single command.

The above examples are merely illustrative of the many applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system adapted to concurrently acquire first data and second data of a patient, the system comprising:
an electrodiagnostic device configured to acquire the first data indicative of voluntary or stimulated electrophysiological signals from a nerve or muscle of the patient;
an ultrasound device configured to apply acoustic energy to the patient and acquire the second data, wherein the second data is indicative of reflected sound from the patient's tissue in response to the application of said acoustic energy into the patient;
a unitary handheld device in electrical communication with the electrodiagnostic device and the ultrasound device, wherein the handheld device is adapted to concurrently apply electrical stimulation to a first anatomical location of the patient and said acoustic energy to a second anatomical location of the patient and wherein the handheld device is further configured to acquire the first data and the second data;
a circuit comprising a clock adapted to generate a time, wherein the circuit is further configured to receive and synchronize the first data and the second data using said time in order to generate third data;
a computing device in data communication with at least one of a) the electrodiagnostic device and the ultrasound device and b) the handheld device, wherein the computing device includes a processor and memory storing a plurality of programmatic instructions which when executed by the processor, configures the processor to:
acquire the first data, the second data, and the third data;
analyze the first data, the second data, and the third data; and
generate one or more graphical user interfaces in order to display the first data, the second data, and the third data based on a selection of at least one of a plurality of clinical applications.

2. The system of claim 1, wherein the third data comprises a unitary file having both the first data and the second data in a same format.

3. The system of claim 1, wherein the electrodiagnostic device is configured to perform at least one of an electromyography study, a nerve conduction study, an evoked potential study, and a repetitive nerve stimulation study.

4. The system of claim 1, wherein the plurality of clinical applications comprises a stimulated M-mode study, an electrodiagnostic triggered M-mode study, a stimulated B-mode study, an electrodiagnostic triggered B-mode study, an ultrasound stimulation study, a repetitive nerve stimulation study, a triggered electrodiagnostic study, video synchronization, and an automatic needle tip identification and 3D visualization of ultrasound and electrodiagnostic data.

5. The system of claim 1, wherein, when the plurality of programmatic instructions is executed by the processor, the processor is further configured to:
automatically acquire at least a portion of the second data indicative of a current depth of a tip of a needle in the patient;
using said second data, cause a display of the current depth of the needle in at least one graphical user interface;
cause a display of a window in the at least one graphical user interface, wherein the window is configured to enhance the needle tip and correlate its location in the ultrasound image with relevant electrodiagnostic data;
store the ultrasound image with needle position and motor unit potential (MUP) data; and
cause a display of said MUP data on the ultrasound image.

6. The system of claim 5, wherein, when the plurality of programmatic instructions is executed by the processor, the processor is further configured to extract from the first data said MUP data and cause the display of said MUP data near the needle tip on the ultrasound image.

7. The system of claim 1, wherein the electrodiagnostic device is an electromyography device.

8. The system of claim 1, wherein the third data comprises data indicative of a test set-up support for generating a muscle scoring table from an electromyography protocol.

9. The system of claim 1, wherein the third data comprises data indicative of navigator support in a graphical user interface.

10. The system of claim 1, wherein, when the plurality of programmatic instructions is executed by the processor, the processor is further configured to cause a display of a toggle control adapted to toggle between a first protocol and a second protocol, wherein the first protocol and the second protocol are different.

11. The system of claim 1, wherein the first protocol corresponds to an electromyography protocol and wherein the second protocol corresponds to an ultrasound protocol.

12. The system of claim 1, wherein, when the plurality of programmatic instructions is executed by the processor, the processor is further configured to generate a dock cine toolbar in the one or more graphical user interfaces.

13. The system of claim 1, wherein the system is adapted to synchronize a cine buffer and an electromyography buffer.

14. The system of claim 1, wherein, when the plurality of programmatic instructions is executed by the processor, the processor is further configured to cause a display two images side-by-side in the one or more graphical user interface, and wherein the two images are indicative of at least two of the first data, the second data and the third data.

15. The system of claim 1, wherein, when the plurality of programmatic instructions is executed by the processor, the processor is further configured to receive a manual selection of two or more images using the one or more graphical user interface, and wherein the two or more images are indicative of at least two of the first data, the second data and the third data.

16. The system of claim 1, wherein, when the plurality of programmatic instructions is executed by the processor, the processor is further configured to create report tokens to support image comparisons, wherein the image is indicative of at least one of the first data, second data or third data.

17. The system of claim 1, wherein, when the plurality of programmatic instructions is executed by the processor, the processor is further configured to:
cause the ultrasound device to access a computing device storing a list of a plurality of open ultrasound scan orders;
cause the ultrasound scan order to be selected from the list of the plurality of open ultrasound scan orders;
add the selected ultrasound scan order to a local study list of the ultrasound device;
acquire one or more images corresponding to the selected ultrasound scan order; and
transmit the one or more acquired images to a storage server.

18. The system of claim 17, wherein the computing device is a DICOM worklist server and wherein the storage server is a DICOM storage server.

19. The system of claim 17, wherein, when the plurality of programmatic instructions is executed by the processor, the processor is further configured to:
receive an ultrasound scan order from a user; and
submit the ultrasound scan order to an electronic health record system.

20. The system of claim 17, wherein, when the plurality of programmatic instructions is executed by the processor, the processor is further configured to display the one or more acquired images and link the one or more acquired images to the ultrasound scan order.

21. A system adapted to concurrently acquire first data and second data of a patient, the system comprising:
an electrodiagnostic device configured to acquire the first data indicative of voluntary or stimulated electrophysiological signals from a nerve or muscle of the patient, wherein the electrodiagnostic device is configured to perform at least one of an electromyography study, a nerve conduction study, an evoked potential study, and a repetitive nerve stimulation study;
an ultrasound device configured to apply acoustic energy to the patient and acquire the second data, wherein the second data is indicative of reflected sound from the patient's tissue in response to the application of said acoustic energy into the patient;
a unitary handheld device in electrical communication with the electrodiagnostic device and the ultrasound device, wherein the handheld device is adapted to concurrently apply electrical stimulation to a first anatomical location of the patient and said acoustic energy to a second anatomical location of the patient and wherein the handheld device is further configured to acquire the first data and the second data;
a circuit comprising a clock adapted to generate a time, wherein the circuit is further configured to receive and synchronize the first data and the second data using said time in order to generate third data, wherein the third data comprises a unitary file having both the first data and the second data in a same format;
a computing device in data communication with at least one of a) the electrodiagnostic device and the ultrasound device and b) the handheld device, wherein the computing device includes a processor and memory storing a plurality of programmatic instructions which when executed by the processor, configures the processor to:
acquire the first data, the second data, and the third data;
analyze the first data, the second data, and the third data; and
generate one or more graphical user interfaces in order to display the first data, the second data, and the third data based on a selection of at least one of a stimulated M-mode study, an electrodiagnostic triggered M-mode study, a stimulated B-mode study, an electrodiagnostic triggered B-mode study, an ultrasound stimulation study, a repetitive nerve stimulation study, a triggered electrodiagnostic study, video synchronization, and an automatic needle tip identification and 3D visualization of ultrasound and electrodiagnostic study.
